SEQUENCE LISTING (101 sequences)

SEQ ID NO: 1 - DNA, 52 bp, Artificial Sequence
  Description of Artificial Sequence Synthetic Oligonucleotide
ctcagctgtcggcaacagtcgccgatcagggcgactcacgaccagactgtgc

SEQ ID NO: 2 - DNA, 52 bp, Artificial Sequence
  Description of Artificial Sequence Synthetic Oligonucleotide
gagtcctgtcggtctcagtcgccgatcagggcgactcgtctgtcatgccgtc

SEQ ID NO: 3 - DNA, 52 bp, Artificial Sequence
  Description of Artificial Sequence Synthetic Oligonucleotide
cctacgatctcacgccagtcgccgatcagggcgactccacacgatgcatgcc

SEQ ID NO: 4 - DNA, 52 bp, Artificial Sequence
  Description of Artificial Sequence Synthetic Oligonucleotide
ctgcacgactgaggtcagtcgccgatcagggcgactggcgttcgcatttggc

SEQ ID NO: 5 - DNA, 52 bp, Artificial Sequence
  Description of Artificial Sequence Synthetic Oligonucleotide
gcatcaggtagccaggagtcgccgatcagggcgactgcttcagctgagaggc

SEQ ID NO: 6 - DNA, 52 bp, Artificial Sequence
  Description of Artificial Sequence Synthetic Oligonucleotide
ctggctcgaaaatgccagtcgccgatcagggcgactccaacctaagccggag

SEQ ID NO: 7 - DNA, 52 bp, Artificial Sequence
  Description of Artificial Sequence Synthetic Oligonucleotide
cgctatgttcaaggtcagtcgccgatcagggcgactcatctgacgttacctg

SEQ ID NO: 8 - DNA, 50 bp, Artificial Sequence
  Description of Artificial Sequence Synthetic Oligonucleotide
cagctgaggaaatcttatacgcctccggccattggctggcgcagcgctct

SEQ ID NO: 9 - DNA, 50 bp, Artificial Sequence
  Description of Artificial Sequence Synthetic Oligonucleotide
aagctgtacatcgtttgagatgaagcctacgatacatgcccggcacagtc

SEQ ID NO: 10 - DNA, 50 bp, Artificial Sequence
  Description of Artificial Sequence Synthetic Oligonucleotide
caggactccagcagggcatgagttagatccattggctggcgcagcgctct

SEQ ID NO: 11 - DNA, 50 bp, Artificial Sequence
  Description of Artificial Sequence Synthetic Oligonucleotide
aagctgtacatcgtttgagattcctaacgtaccaacgcacgggacggcat

SEQ ID NO: 12 - DNA, 50 bp, Artificial Sequence
  Description of Artificial Sequence Synthetic Oligonucleotide
atcgtaggtctgccctgacttctatgccccattggctggcgcagcgctct

SEQ ID NO: 13 - DNA, 50 bp, Artificial Sequence
  Description of Artificial Sequence Synthetic Oligonucleotide
aagctgtacatcgtttgagatcagaggacaacttcctacgtaggcatgca

SEQ ID NO: 14 - DNA, 50 bp, Artificial Sequence
  Description of Artificial Sequence Synthetic Oligonucleotide
tcgtgcagtggtcggattctcaactcgtccattggctggcgcagcgctct

SEQ ID NO: 15 - DNA, 50 bp, Artificial Sequence
  Description of Artificial Sequence Synthetic Oligonucleotide
aagctgtacatcgtttgagattaagatgaagataggccacccgccaaatg

SEQ ID NO: 16 - DNA, 50 bp, Artificial Sequence
  Description of Artificial Sequence Synthetic Oligonucleotide
cctgatgcctctttcaacggcgtattaagcattggctggcgcagcgctct

SEQ ID NO: 17 - DNA, 50 bp, Artificial Sequence
  Description of Artificial Sequence Synthetic Oligonucleotide
aagctgtacatcgtttgagatttcgtctgcatgagtgatgtcgcctctca

SEQ ID NO: 18 - DNA, 57 bp, Artificial Sequence
  Description of Artificial Sequence Synthetic Oligonucleotide
cagctgagtttttttgaaatcttatacgcctccggccattggctggcgcagcgctct

SEQ ID NO: 19 - DNA, 57 bp, Artificial Sequence
  Description of Artificial Sequence Synthetic Oligonucleotide
aagctgtacatcgtttgagatgaagcctacgatacatgcccgtttttttgcacagtc

SEQ ID NO: 20 - DNA, 57 bp, Artificial Sequence
  Description of Artificial Sequence Synthetic Oligonucleotide
caggactctttttttcagcagggcatgagttagatccaagtcaactcatgcagagac

SEQ ID NO: 21 - DNA, 57 bp, Artificial Sequence
  Description of Artificial Sequence Synthetic Oligonucleotide
ttgctcaccagaacgagtagttcctaacgtaccaacgcacggtttttttgacggcat

SEQ ID NO: 22 - DNA, 57 bp, Artificial Sequence
  Description of Artificial Sequence Synthetic Oligonucleotide
atcgtaggttttttttctgccctgacttctatgcccgcatgaactatagactgcgtc

SEQ ID NO: 23 - DNA, 57 bp, Artificial Sequence
  Description of Artificial Sequence Synthetic Oligonucleotide
gagcacactagcacactcatccagaggacaacttcctacgtatttttttggcatgca

SEQ ID NO: 24 - DNA, 57 bp, Artificial Sequence
  Description of Artificial Sequence Synthetic Oligonucleotide
tcgtgcagttttttttggtcggattctcaactcgtcgatgaataaggcttgcccggt

SEQ ID NO: 25 - DNA, 57 bp, Artificial Sequence
  Description of Artificial Sequence Synthetic Oligonucleotide
gatgaataaggcttgccctgttaagatgaagataggccaccctttttttgccaaatg

SEQ ID NO: 26 - DNA, 57 bp, Artificial Sequence
  Description of Artificial Sequence Synthetic Oligonucleotide
cctgatgctttttttctctttcaacggcgtattaagacggctacagaggctttgagg

SEQ ID NO: 27 - DNA, 57 bp, Artificial Sequence
  Description of Artificial Sequence Synthetic Oligonucleotide
ctatttttgcggatggcttagttcgtctgcatgagtgatgtctttttttgcctctca

SEQ ID NO: 28 - DNA, 57 bp, Artificial Sequence
  Description of Artificial Sequence Synthetic Oligonucleotide
cgagccagtttttttcggatcgtactatggttgccttagcatcggaacgagggtaag

SEQ ID NO: 29 - DNA, 57 bp, Artificial Sequence
  Description of Artificial Sequence Synthetic Oligonucleotide
tcgactgtaccaagagccatccttgaaccaccacagcttacgtttttttctccggct

SEQ ID NO: 30 - DNA, 57 bp, Artificial Sequence
  Description of Artificial Sequence Synthetic Oligonucleotide
acatagcgtttttttctggaggtggcatcctactgttgcgccgacaatgacaactac

SEQ ID NO: 31 - DNA, 57 bp, Artificial Sequence
  Description of Artificial Sequence Synthetic Oligonucleotide
cgattgtagctgaggcttgctagtacggtgtctggaagtttctttttttcaggtaac

SEQ ID NO: 32 - DNA, 8 bp, Artificial Sequence
  Description of Artificial Sequence Synthetic Oligonucleotide
gttgccga

SEQ ID NO: 33 - DNA, 8 bp, Artificial Sequence
  Description of Artificial Sequence Synthetic Oligonucleotide
tggtcgtg

SEQ ID NO: 34 - DNA, 8 bp, Artificial Sequence
  Description of Artificial Sequence Synthetic Oligonucleotide
gagaccga

SEQ ID NO: 35 - DNA, 8 bp, Artificial Sequence
  Description of Artificial Sequence Synthetic Oligonucleotide
gacagacg

SEQ ID NO: 36 - DNA, 8 bp, Artificial Sequence
  Description of Artificial Sequence Synthetic Oligonucleotide
ggcgtgag

SEQ ID NO: 37 - DNA, 8 bp, Artificial Sequence
  Description of Artificial Sequence Synthetic Oligonucleotide
tcgtgtgg

SEQ ID NO: 38 - DNA, 8 bp, Artificial Sequence
  Description of Artificial Sequence Synthetic Oligonucleotide
gacctcag

SEQ ID NO: 39 - DNA, 8 bp, Artificial Sequence
  Description of Artificial Sequence Synthetic Oligonucleotide
cgaacgcc

SEQ ID NO: 40 - DNA, 8 bp, Artificial Sequence
  Description of Artificial Sequence Synthetic Oligonucleotide
cctggcta

SEQ ID NO: 41 - DNA, 8 bp, Artificial Sequence
  Description of Artificial Sequence Synthetic Oligonucleotide
gctgaagc

SEQ ID NO: 42 - DNA, 8 bp, Artificial Sequence
  Description of Artificial Sequence Synthetic Oligonucleotide
ggcatttt

SEQ ID NO: 43 - DNA, 8 bp, Artificial Sequence
  Description of Artificial Sequence Synthetic Oligonucleotide
taggttgg

SEQ ID NO: 44 - DNA, 8 bp, Artificial Sequence
  Description of Artificial Sequence Synthetic Oligonucleotide
gaccttga

SEQ ID NO: 45 - DNA, 8 bp, Artificial Sequence
  Description of Artificial Sequence Synthetic Oligonucleotide
gtcagatg

SEQ ID NO: 46 - DNA, 25 bp, Artificial Sequence
  Description of Artificial Sequence Synthetic Oligonucleotide
gcaccgggagggagggagggctttt

SEQ ID NO: 47 - DNA, 15 bp, Artificial Sequence
  Description of Artificial Sequence Synthetic Oligonucleotide
ttcctctaccaccta

SEQ ID NO: 48 - DNA, 15 bp, Artificial Sequence
  Description of Artificial Sequence Synthetic Oligonucleotide
taggtggtagaggaa

SEQ ID NO: 49 - DNA, 45 bp, Artificial Sequence
  Description of Artificial Sequence Synthetic Oligonucleotide
gatctaactcatgccctgctgtttcgggcatgtatcgtaggcttc

SEQ ID NO: 50 - DNA, 45 bp, Artificial Sequence
  Description of Artificial Sequence Synthetic Oligonucleotide
gggcatagaagtcagggcagatttccgtgcgttggtacgttagga

SEQ ID NO: 51 - DNA, 45 bp, Artificial Sequence
  Description of Artificial Sequence Synthetic Oligonucleotide
gacgagttgagaatccgaccattttacgtaggaagttgtcctctg

SEQ ID NO: 52 - DNA, 45 bp, Artificial Sequence
  Description of Artificial Sequence Synthetic Oligonucleotide
cttaatacgccgttgaaagagtttgggtggcctatcttcatctta

SEQ ID NO: 53 - DNA, 45 bp, Artificial Sequence
  Description of Artificial Sequence Synthetic Oligonucleotide
gccggaggcgtataagatttctttgacatcactcatgcagacgaa

SEQ ID NO: 54 - DNA, 45 bp, Artificial Sequence
  Description of Artificial Sequence Synthetic Oligonucleotide
gccggaggcgtataagatttctttcgtaagctgtggtggttcaag

SEQ ID NO: 55 - DNA, 45 bp, Artificial Sequence
  Description of Artificial Sequence Synthetic Oligonucleotide
gccggaggcgtataagatttctttgaaacttccagacaccgtact

SEQ ID NO: 56 - DNA, 49 bp, Artificial Sequence
  Description of Artificial Sequence Synthetic Oligonucleotide
atctcaaacgatgtacagctttttttttagagcgctgcgccagccaatg

SEQ ID NO: 57 - DNA, 49 bp, Artificial Sequence
  Description of Artificial Sequence Synthetic Oligonucleotide
actactcgttctggtgagcaatttttttgtctctgcatgagttgacttg

SEQ ID NO: 58 - DNA, 49 bp, Artificial Sequence
  Description of Artificial Sequence Synthetic Oligonucleotide
gatgagtgtgctagtgtgctctttttttgacgcagtctatagttcatgc

SEQ ID NO: 59 - DNA, 49 bp, Artificial Sequence
  Description of Artificial Sequence Synthetic Oligonucleotide
acagggcaagccttattcatctttttttaccgggcaagccttattcatc

SEQ ID NO: 60 - DNA, 49 bp, Artificial Sequence
  Description of Artificial Sequence Synthetic Oligonucleotide
ctaagccatccgcaaaaatagtttttttcctcaaagcctctgtagccgt

SEQ ID NO: 61 - DNA, 49 bp, Artificial Sequence
  Description of Artificial Sequence Synthetic Oligonucleotide
gatggctcttggtacagtcgatttttttcttaccctcgttccgatgcta

SEQ ID NO: 62 - DNA, 49 bp, Artificial Sequence
  Description of Artificial Sequence Synthetic Oligonucleotide
agcaagcctcagctacaatcgtttttttgtagttgtcattgtcggcgca

SEQ ID NO: 63 - DNA, 11 bp, Artificial Sequence
  Description of Artificial Sequence Synthetic Oligonucleotide
cagctgagttt

SEQ ID NO: 64 - DNA, 8 bp, Artificial Sequence
  Description of Artificial Sequence Synthetic Oligonucleotide
gcacagtc

SEQ ID NO: 65 - DNA, 16 bp, Artificial Sequence
  Description of Artificial Sequence Synthetic Oligonucleotide
atcgtagggacggcat

SEQ ID NO: 66 - DNA, 16 bp, Artificial Sequence
  Description of Artificial Sequence Synthetic Oligonucleotide
tcgtgcagggcatgca

SEQ ID NO: 67 - DNA, 15 bp, Artificial Sequence
  Description of Artificial Sequence Synthetic Oligonucleotide
cctgatgcgccaaat

SEQ ID NO: 68 - DNA, 16 bp, Artificial Sequence
  Description of Artificial Sequence Synthetic Oligonucleotide
caggactcgcacagtc

SEQ ID NO: 69 - DNA, 58 bp, Artificial Sequence
  Description of Artificial Sequence Synthetic Oligonucleotide
gttgccgacagctgaggaaatcttatacgcctccggccattggctggcgcagcgctct

SEQ ID NO: 70 - DNA, 58 bp, Artificial Sequence
  Description of Artificial Sequence Synthetic Oligonucleotide
aagctgtacatcgtttgagatgaagcctacgatacatgcccggcacagtctggtcgtg

SEQ ID NO: 71 - DNA, 58 bp, Artificial Sequence
  Description of Artificial Sequence Synthetic Oligonucleotide
gagaccgacaggactccagcagggcatgagttagatccattggctggcgcagcgctct

SEQ ID NO: 72 - DNA, 58 bp, Artificial Sequence
  Description of Artificial Sequence Synthetic Oligonucleotide
aagctgtacatcgtttgagattcctaacgtaccaacgcacgggacggcatgacagacg

SEQ ID NO: 73 - DNA, 58 bp, Artificial Sequence
  Description of Artificial Sequence Synthetic Oligonucleotide
ggcgtgagatcgtaggtctgccctgacttctatgccccattggctggcgcagcgctct

SEQ ID NO: 74 - DNA, 58 bp, Artificial Sequence
  Description of Artificial Sequence Synthetic Oligonucleotide
aagctgtacatcgtttgagatcagaggacaacttcctacgtaggcatgcatcgtgtgg

SEQ ID NO: 75 - DNA, 58 bp, Artificial Sequence
  Description of Artificial Sequence Synthetic Oligonucleotide
gacctcagtcgtgcagtggtcggattctcaactcgtccattggctggcgcagcgctct

SEQ ID NO: 76 - DNA, 58 bp, Artificial Sequence
  Description of Artificial Sequence Synthetic Oligonucleotide
aagctgtacatcgtttgagattaagatgaagataggccacccgccaaatgcgaacgcc

SEQ ID NO: 77 - DNA, 58 bp, Artificial Sequence
  Description of Artificial Sequence Synthetic Oligonucleotide
cctggctacctgatgcctctttcaacggcgtattaagcattggctggcgcagcgctct

SEQ ID NO: 78 - DNA, 58 bp, Artificial Sequence
  Description of Artificial Sequence Synthetic Oligonucleotide
aagctgtacatcgtttgagatttcgtctgcatgagtgatgtcgcctctcagctgaagc

SEQ ID NO: 79 - DNA, 65 bp, Artificial Sequence
  Description of Artificial Sequence Synthetic Oligonucleotide
gttgccgacagctgagtttttttgaaatcttatacgcctccggccattggctggcgcagcgctct

SEQ ID NO: 80 - DNA, 65 bp, Artificial Sequence
  Description of Artificial Sequence Synthetic Oligonucleotide
aagctgtacatcgtttgagatgaagcctacgatacatgcccgtttttttgcacagtctggtcgtg

SEQ ID NO: 81 - DNA, 65 bp, Artificial Sequence
  Description of Artificial Sequence Synthetic Oligonucleotide
gagaccgacaggactctttttttcagcagggcatgagttagatccaagtcaactcatgcagagac

SEQ ID NO: 82 - DNA, 65 bp, Artificial Sequence
  Description of Artificial Sequence Synthetic Oligonucleotide
ttgctcaccagaacgagtagttcctaacgtaccaacgcacggtttttttgacggcatgacagacg

SEQ ID NO: 83 - DNA, 65 bp, Artificial Sequence
  Description of Artificial Sequence Synthetic Oligonucleotide
ggcgtgagatcgtaggttttttttctgccctgacttctatgcccgcatgaactatagactgcgtc

SEQ ID NO: 84 - DNA, 65 bp, Artificial Sequence
  Description of Artificial Sequence Synthetic Oligonucleotide
gagcacactagcacactcatccagaggacaacttcctacgtatttttttggcatgcatcgtgtgg

SEQ ID NO: 85 - DNA, 65 bp, Artificial Sequence
  Description of Artificial Sequence Synthetic Oligonucleotide
gacctcagtcgtgcagttttttttggtcggattctcaactcgtcgatgaataaggcttgcccggt

SEQ ID NO: 86 - DNA, 65 bp, Artificial Sequence
  Description of Artificial Sequence Synthetic Oligonucleotide
gatgaataaggcttgccctgttaagatgaagataggccaccctttttttgccaaatgcgaacgcc

SEQ ID NO: 87 - DNA, 65 bp, Artificial Sequence
  Description of Artificial Sequence Synthetic Oligonucleotide
cctggctacctgatgctttttttctctttcaacggcgtattaagacggctacagaggctttgagg

SEQ ID NO: 88 - DNA, 65 bp, Artificial Sequence
  Description of Artificial Sequence Synthetic Oligonucleotide
ctatttttgcggatggcttagttcgtctgcatgagtgatgtctttttttgcctctcagctgaagc

SEQ ID NO: 89 - DNA, 65 bp, Artificial Sequence
  Description of Artificial Sequence Synthetic Oligonucleotide
ggcattttcgagccagtttttttcggatcgtactatggttgccttagcatcggaacgagggtaag

SEQ ID NO: 90 - DNA, 65 bp, Artificial Sequence
  Description of Artificial Sequence Synthetic Oligonucleotide
tcgactgtaccaagagccatccttgaaccaccacagcttacgtttttttctccggcttaggttgg

SEQ ID NO: 91 - DNA, 65 bp, Artificial Sequence
  Description of Artificial Sequence Synthetic Oligonucleotide
gaccttgaacatagcgtttttttctggaggtggcatcctactgttgcgccgacaatgacaactac

SEQ ID NO: 92 - DNA, 65 bp, Artificial Sequence
  Description of Artificial Sequence Synthetic Oligonucleotide
cgattgtagctgaggcttgctagtacggtgtctggaagtttctttttttcaggtaacgtcagatg

SEQ ID NO: 93 - DNA, 19 bp, Artificial Sequence
  Description of Artificial Sequence Synthetic Oligonucleotide
gttgccgacagctgagttt

SEQ ID NO: 94 - DNA, 16 bp, Artificial Sequence
  Description of Artificial Sequence Synthetic Oligonucleotide
gcacagtcgctgaagc

SEQ ID NO: 95 - DNA, 32 bp, Artificial Sequence
  Description of Artificial Sequence Synthetic Oligonucleotide
ggcgtgagatcgtagggacggcatgacagacg

SEQ ID NO: 96 - DNA, 32 bp, Artificial Sequence
  Description of Artificial Sequence Synthetic Oligonucleotide
gacctcagtcgtgcagggcatgcatcgtgtgg

SEQ ID NO: 97 - DNA, 31 bp, Artificial Sequence
  Description of Artificial Sequence Synthetic Oligonucleotide
cctggctacctgatgcgccaaatcgaacgcc

SEQ ID NO: 98 - DNA, 32 bp, Artificial Sequence
  Description of Artificial Sequence Synthetic Oligonucleotide
gagaccgacaggactcgcacagtctggtcgtg

SEQ ID NO: 99 - DNA, 22 bp, Artificial Sequence
  Description of Artificial Sequence Synthetic Oligonucleotide
caggttatggcactgtcacgat

SEQ ID NO: 100 - DNA, 22 bp, Artificial Sequence
  Description of Artificial Sequence Synthetic Oligonucleotide
ccatctgcagcaacaccatctc

SEQ ID NO: 101 - DNA, 23 bp, Artificial Sequence
  Description of Artificial Sequence Synthetic Oligonucleotide
ctctccgagaacaggcctcgact

(19) United States
(12) Patent Application Publication
(10) Pub. No.: US 2023/0212643 A1
(43) Pub. Date: Jul. 6, 2023

(54) DNA NANOARCHITECTURES FOR PATTERN-RECOGNIZED TARGETING OF DISEASES

(71) Applicant: RENSSELAER POLYTECHNIC INSTITUTE, Troy (US)

(72) Inventors: Xing WANG, Champaign, IL (US); Seok-Joon KWON, Niskayuna, NY (US); Jonathan Seth DORDICK, Schenectady, NY (US); Robert John LINHARDT, Albany, NY (US); Paul S. KWON, Niskayuna, NY (US)

(73) Assignee: RENSSELAER POLYTECHNIC INSTITUTE, Troy, NY (US)

(21) Appl. No.: 17/612,000

(22) PCT Filed: May 18, 2020
(86) PCT No.: PCT/US20/33398

§ 371 Date: Nov. 17, 2021

Related U.S. Application Data
(60) Provisional application No. 63/022,644, filed on May 11, 2020.
(60) Provisional application No. 62/849,327, filed on May 17, 2019.

(51) Int. Cl. C12Q 1/68; C12Q 1/70; C12N 15/115

(52) U.S. Cl. CPC C12Q 1/68; C12Q 1/701; C12N 15/115; C12N 2310/16; C12Q 2600/16

(57) ABSTRACT

The oligonucleotide nanostructures enable pattern-recognized targeting of diseases, particularly useful as high-specificity detectors and inhibitors of viruses and toxins, such as for Dengue virus particles. The nanostructures include an oligonucleotide scaffold with a plurality of binders arranged in a pattern conforming to a plurality of surface epitopes of a target disease. Binding of the scaffolds to these surface epitopes has been shown to have inhibitory effects against the target disease. The scaffolds can also include functional domains that activate upon target binding. Assembly of the scaffolds can be achieved via annealing of separate oligonucleotide segments of predetermined length and sequence, which also advantageously define locations of binding domains in the resulting structure. This approach provides precise control over the spacing and orientation of epitope binding sites in the scaffold.

### CROSS REFERENCE TO RELATED APPLICATION(S)

This application is a national stage filing of International Patent Application No. PCT/US2020/033398, filed May 18, 2020, which claims the benefit of U.S. Provisional Application Nos. 62/022,644, filed May 11, 2020, and 62/849,327, filed May 17, 2019, which are incorporated by reference as if disclosed herein in their entireties.

### INCORPORATION OF SEQUENCE LISTING

The contents of the text file named Sequences_replacement_ST25.txt, which was created on Jul. 12, 2022 and is 24.7 KB is size, are hereby incorporated by reference in their entireties.

### BACKGROUND

Prevention or treatment of viral infection typically relies on neutralizing antibodies (NAbs) against target epitopes on the viral surface. Production of NAbs can be triggered by vaccines or active viruses that invade hosts, though there are significant drawbacks to this method of treatment. Currently available vaccines or immunological memory from prior infections may not provide protection against epidemics as a result of escape-variant viruses, which arise mainly from genetic drift. NAbs can also induce antibody-dependent enhancement of infection (e.g., dengue). Finally, there remains no approved therapeutics or vaccines available for treatment or prevention of many World Health Organization priority “blueprint-viruses” such as Zika and HIV. In viral detection, the most common diagnostic approaches employ virus isolation, antigen capture immunoassays and molecular diagnostic tests, which are typically time-consuming, expensive, and require a sophisticated clinical laboratory setup and technical expertise. A recently reported toehold switch-based RNA detection scheme offers a solution for preparing viral sensors. However, this strategy still involves a nucleic acid sequence-based amplification (NASBA) reaction, which requires time-consuming thermal cycling. Avoiding a PCR-based method would enable facile antiviral prevention, treatment and direct sensing that may enable timely detection and control of pandemic outbreaks within surveillance and diagnostic networks.

Infectious diseases, including viruses and toxins, present a unique pattern of antigens on their surfaces. Existing weakly binding ligands (or “binders”) that interact with these epitopes can be linked to a scaffold affording strong multivalent binders/inhibitors. Several scaffolds including polymers, dendrimers, nanofibers, nanoparticles, and lipid nanoemulsions have been used to arrange weak binders sporadically to match the average spacing of epitopes. However, some viruses, such as Dengue virus, present complex geometric patterns that cannot be addressed by existing scaffolds because they lack sufficiently precise ligand spacing or provide limited control over the scaffold shape and ligand valency. Furthermore, these previous synthetic scaffolds have shown some toxicity.

### SUMMARY

Accordingly, some embodiments of the present disclosure relates to a structure for pattern-recognized targeting of diseases. In some embodiments, an oligonucleotide scaffold including a plurality of binder insertion regions, wherein the binder insertion regions are arranged in a pattern conforming to a plurality of epitopes of a target, and a plurality of binders incorporated into the binder insertion regions, wherein the binders are configured to bind at least one of the plurality of epitopes. In some embodiments, one or more functional domains having an activity, wherein the activity includes inhibition, signaling, therapeutic, or combinations thereof. In some embodiments, the functional domains exhibit a first activity when unbound and a second activity when bound to the plurality of epitopes. In some embodiments, the functional domains include a hybridized fluorophore and a quencher, wherein the quencher inactivates the fluorophore as the first activity, and wherein the quencher separates from the fluorophore upon binding of the plurality of binders to generate a detectable fluorescent signal as the second activity. In some embodiments, the binders include peptides, aptamers, oligosaccharides, small molecules, or combinations thereof. In some embodiments, the target is Dengue virus, Zika virus, influenza virus, adenovirus, bacterial toxin, or combinations thereof. In some embodiments, the scaffold includes one or more interior scaffold segments and one or more exterior edge segments. In some embodiments, the one or more exterior edge segments include SEQ. ID. NO.: 8-45S, 49-68A, or combinations thereof. In some embodiments, the interior scaffold segments include a stem-loop structure. In some embodiments, the interior scaffold segments include SEQ. ID. NO.: 1-7, or combinations thereof. In some embodiments, five aptamers attached to interior scaffold segments, wherein the aptamers attach to the interior scaffold segments at the 3′ ends of the interior scaffold segments. In some embodiments, five aptamers attached to exterior edge segments, wherein the aptamers attach to the exterior edge segments at the 3′ ends of the exterior edge segments. In some embodiments, the interior scaffold segments and exterior edge segments are arranged in a 3D pattern.

Some embodiments of the present disclosure relate to a method of making a structure for pattern-recognized targeting of diseases. In some embodiments, the method includes preparing a plurality of binders configured to bind at least one of a plurality of epitopes of a target. In some embodiments, the method includes identifying a spatial pattern of the plurality of epitopes. In some embodiments, the method includes preparing an oligonucleotide scaffold including a plurality of binder insertion regions, wherein the binder insertion regions are arranged to correspond to the spatial pattern. In some embodiments, the method includes incorporating one or more binders into the binder insertion regions. In some embodiments, the method includes incorporating one or more functional domains into the oligonucleotide scaffold, wherein the functional domains have an activity, wherein the activity includes inhibition, signaling, therapeutic, or combinations thereof.

In some embodiments, preparing an oligonucleotide scaffold including a plurality of binder insertion regions includes annealing one or more interior scaffold segments with one or more exterior edge segments, wherein the interior scaffold segments include a stem-loop structure. In some embodiments, five aptamers are annealed to the interior scaffold segments, wherein the aptamers attach to the interior scaffold segments at the 3′ ends of the interior scaffold segments. In some embodiments, five aptamers are annealed to exterior edge segments, wherein the aptamers attach to the exterior edge segments at the 3′ ends of the exterior edge segments.

Some embodiments of the present disclosure relate to a method of targeting a disease in a patient. In some embodiments, the method includes identifying a spatial pattern of a plurality of epitopes on a surface of the disease. In some embodiments, the method includes preparing an oligonucleotide scaffold including, annealing one or more interior scaffold segments with one or more exterior edge segments to define a structure including a plurality of binder insertion regions, wherein the binder insertion regions are arranged to correspond to the spatial pattern, annealing a plurality of aptamers at the binder insertion regions, and annealing one or more functional domains to the interior scaffold segments, the one or more exterior edge segments, or combinations thereof, wherein the functional domains have an activity, wherein the activity includes inhibition, signaling, therapeutic, or combinations thereof, administering an amount of the oligonucleotide scaffold to the patient, and measuring a level of activity of the functional domains. In some embodiments, the target is configured to bind to SEQ. ID. NO.: 46.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic representation of a pattern-recognizing oligonucleotide scaffold according to some embodiments of the present disclosure;

FIG. 2 is a schematic representation of surface epitopes of Dengue virus;

FIG. 3A is an atomic force microscopy image of a pattern-recognizing oligonucleotide scaffold according to some embodiments of the present disclosure;

FIG. 3B is an image of polyacrylamide gel electrophoresis analysis of pattern-recognizing oligonucleotide scaffolds according to some embodiments of the present disclosure;

FIG. 4A is a chart portraying detection of Dengue virus using pattern-recognizing oligonucleotide scaffolds according to some embodiments of the present disclosure;

FIG. 4B is a chart portraying preferential binding of Dengue virus using pattern-recognizing oligonucleotide scaffolds according to some embodiments of the present disclosure;

FIG. 5A is a chart comparing Dengue virus inhibition by various pattern-recognizing oligonucleotide scaffolds according to some embodiments of the present disclosure;

FIG. 5B is a chart comparing Dengue virus inhibition by various pattern-recognizing oligonucleotide scaffolds according to some embodiments of the present disclosure;

FIG. 6 is a chart of a method of making a structure for pattern-recognized targeting of diseases according to some embodiments of the present disclosure; and

FIG. 7 is a chart of a method of targeting a disease in a patient according to some embodiments of the present disclosure.

### DETAILED DESCRIPTION

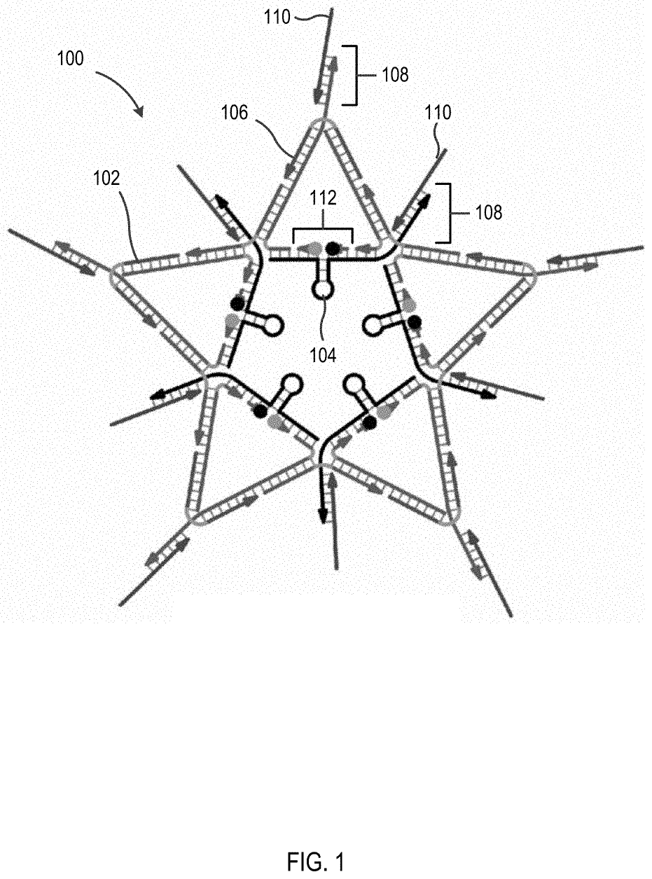

Referring now to FIG. 1, in some embodiments, the system of the present disclosure is directed to a structure 100 for pattern-recognized targeting of diseases. In some embodiments, structure 100 includes an oligonucleotide scaffold 102. In some embodiments, oligonucleotide scaffold 102 includes one or more interior scaffold segments 104. In some embodiments, oligonucleotide scaffold 102 includes one or more exterior edge segments 106. In some embodiments, oligonucleotide scaffold 102 includes a plurality of interior scaffold segments 104. In some embodiments, one or more interior scaffold segments 104 include a stem-loop structure. In some embodiments, oligonucleotide scaffold 102 includes a plurality of exterior edge segments 106. In some embodiments, one or more exterior edge segments 106 include a stem-loop structure. In some embodiments, interior scaffold segments 104 are bound to exterior scaffold segments 106, other interior scaffold segments, or combinations thereof. In some embodiments, exterior edge segments 106 are bound to interior scaffold segments 104, other exterior edge segments, or combinations thereof. In some embodiments, at least some segments of oligonucleotide scaffold 102 are bound together via annealing of complementary oligonucleotides in the respective segments, as will be discussed in greater detail below. In some embodiments, oligonucleotide scaffold 102 is a multi-dimensional structure where exterior edge segments 106 substantially surround interior scaffold segments 104. In some embodiments, oligonucleotide scaffold 102 is arranged in a 1D pattern, i.e., linearly. In some embodiments, oligonucleotide scaffold 102 is arranged in a 2D pattern. In some embodiments, oligonucleotide scaffold 102 is arranged in a 3D pattern. In some embodiments, oligonucleotide scaffold 102 is biocompatible so as to enable use, e.g., infectious disease inhibition, detection, and treatment, in vivo.

In some embodiments, oligonucleotide scaffold 102 includes a plurality of binder insertion regions 108. In some embodiments, binder insertion regions 108 are included in interior scaffold segments 104, exterior edge segments 106, or combinations thereof. In some embodiments, binder insertion regions 108 are arranged in a predetermined spatial pattern. In some embodiments, the spatial pattern of binder insertion regions 108 are determined by the combination of interior scaffold segments 104 and exterior edge segments 106, i.e., these segments are sized, sequenced, and provided at appropriate molar ratios to form a multi-dimensional structure pattern that produces a desired distance between and orientation of the binder insertion regions, as will be discussed in greater detail below.

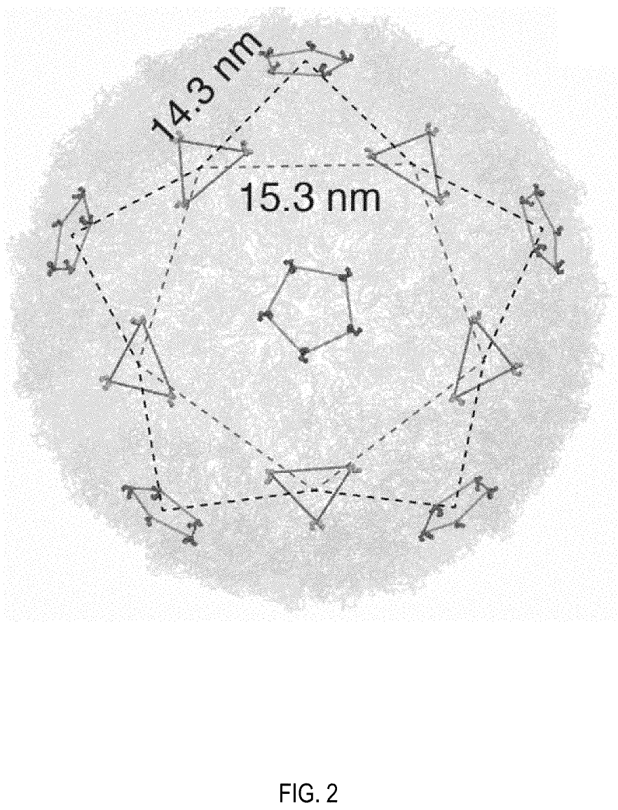

In this way, oligonucleotide scaffold 102 can be rationally designed to precisely arrange a plurality of binder insertion regions 108 in 1D, 2D, or 3D space. In some embodiments, binder insertion regions 108 are arranged in a pattern conforming to a plurality of epitopes of a target. In some embodiments, the epitopes are arranged on a surface of the target. In some embodiments, the target is an infectious disease, e.g., virus particle, bacteria, etc. In some embodiments, the virus is Dengue virus, Zika virus, influenza virus, adenovirus, etc. In some embodiments, the bacteria is Bacillus anthracis, etc. In some embodiments, the epitope includes a surface protein, multi-protein structure, polysaccharide, oligonucleotide, small molecule, or combinations thereof. Referring now to FIG. 2, by way of example, Dengue virus (DENV, Type-2 strain) is an enveloped arbovirus from the Flaviviridae family. The viral diameter of DENV is 50 nm and vector distances were measured using PyMOL. DENV includes a viral surface epitope envelope, protein binding-domain III (ED3), that is organized into a complex icosahedral shape with alternating clusters of three or five ED3 sites. The distance between adjacent trivalent-trivalent clusters was 15.1 nm while the distance between adjacent trivalent-pentavalent clusters was 14.1 nm. The following equation was used to calculate the orthodromic distances to design a scaffold 102 for DENV:

$$orthodromic distance = viral diameter * {sin}^{- 1} ( \frac{cluster inter - distance}{viral diameter} )$$

The orthodromic center-to-center distances between trivalent-trivalent and trivalent-pentavalent clusters were 15.3 nm and 14.3 nm, respectively. This represents a challenging pattern to target using previous inhibitor design strategies.

By connecting the clusters of ED3 sites with lines, it is revealed that a 5-pointed star-shape would provide an advantageous scaffold that mirrors the global icosahedral pattern of ED3 clusters and also matches its local inter-cluster spacing having a surface distance between the adjacent, alternating ED3 clusters of 15-nm. Thus, in this exemplary embodiment, oligonucleotide scaffold 102 specific to DENV can be composed of suitably sized and arranged interior scaffold segments 104 and exterior edge segments 106 to form a 5-pointed star that defines binder insertion regions 108 corresponding to the alternating clusters of three/five ED3 sites.

In some embodiments, segments 104 and 106 of oligonucleotide scaffold 102 have any suitable sequence or composition to form a stable structure that positions the desired number of binder insertion regions 108 in the desired spatial arrangement. In some embodiments, interior scaffold segments 104 include SEQ. ID. NOs.: 1-7, or combinations thereof. In some embodiments, exterior edge segments 106 include SEQ. ID. NOs.: 8-31, 49-68, 69-92 (also referred to as 8A-31A), 93-98 (also referred to as 63A-68A), or combinations thereof. As used herein, sequence identification numbers are meant to include the identified sequence, as well as functional equivalents thereof, which can include sequence and/or compositional additions, deletions, or substitutions that do not substantially affect the structure, function, or binding characteristics of the sequence, either for other segments during formation of scaffold 102 or other structures, e.g., target epitopes. In some embodiments, the sequence identification numbers are meant to include the identified sequences, as well as those with greater than 85%, greater than 90%, or greater than 95% sequence identity with the identified sequences.

Referring again to FIG. 1, in some embodiments, oligonucleotide scaffold 102 includes a plurality of binders 110 incorporated into binder insertion regions 108. In some embodiments, binders 110 include peptides, aptamers, oligosaccharides, small molecules, or combinations thereof. In one exemplary embodiment of an aptamer binder, binder 110 includes SEQ. ID. NO.: 46. In some embodiments, at least some binders 110 are incorporated into oligonucleotide scaffold 102 via annealing with complementary oligonucleotides in interior scaffold segments 104, exterior edge segments 106, or combinations thereof, as will be discussed in greater detail below. In some embodiments, binders 110 include a nucleotide sequence that overlaps with a complimentary sequence in one of interior scaffold segments 104 or exterior edge segments 106. In one exemplary embodiment of such an overlap, an aptamer binder, e.g., SEQ. ID. NO.: 46, further includes SEQ. ID. NO.: 48 which anneals to complimentary sequence SEQ. ID. NO.: 47 appended to one or more interior scaffold segments 104 or exterior edge segments 106 in scaffold 102, e.g., SEQ. ID. NOs.: 1-7. Binders 110 can be thus incorporated into scaffold 102 at predetermined regions 108 (in this example, at sequence overlap regions of the binders and the segments). In some embodiments, binders 110 are incorporated into a plurality of oligonucleotide scaffolds 102. In some embodiments, binders 110 are configured to bind the epitopes of a target. In some embodiments, binders 110 are configured to bind at least one of a plurality of epitopes of a target. In some embodiments, binders 110 each bind the same epitope. In some embodiments, binders 110 bind two or more different epitopes. In some embodiments, the binding of binders 110 to the epitopes of the target include covalent bonds, hydrogen bonds, ionic bonds, van der Waals forces, or combinations thereof. In some embodiments, binding of oligonucleotide scaffold 102 to a target substantially inactivates the target, e.g., binding of the scaffold to the surface of a virus inhibits the activity of the virus, as will be discussed in greater detail below. In some embodiments, binding of binders 110 to a target epitope results in a conformational change in oligonucleotide scaffold 102. By way of example, in some embodiments, one or more segments of oligonucleotide scaffold 102, e.g., segments 104 and/or 106, include a stem-loop structure. In some embodiments, binding of binders 110 applies a stretching force to segments of oligonucleotide scaffold 102, causing the stem-loop structures to unzip and adopt a more linear conformation, which in turn causes the overall structure of the scaffold to expand.

In some embodiments, oligonucleotide scaffold 102 includes one or more functional domains 112. In some embodiments, functional domains 112 are included in interior scaffold segments 104, exterior edge segments 106, or combinations thereof. In some embodiments, functional domains 112 include SEQ. ID. NOs.: 32-45, or combinations thereof. In some embodiments, one or more proteins, multi-protein structures, polysaccharides, oligonucleotides, small molecules, or combinations thereof are appended to SEQ. ID. NOs.: 32-45. In some embodiments, functional domains 112 are biocompatible so as to enable infectious disease inhibition, detection, and treatment in vivo. In some embodiments, oligonucleotide scaffold 102 includes a plurality of functional domains 112. In some embodiments, functional domains 112 have an activity including inhibition, signaling, therapeutic, or combinations thereof.

In some embodiments, functional domains 112 exhibit a first activity when oligonucleotide scaffold 102 is unbound and a second activity when oligonucleotide scaffold 102 is bound, e.g., to the surface of a target via a plurality of epitopes. In some embodiments, functional domains 112 include a hybridized fluorophore and a quencher. In these embodiments, when oligonucleotide scaffold 102 is not bound to a target, the quencher is hybridized with the fluorophore and inactivates it. However, upon binding of oligonucleotide scaffold 102 with a target, a conformational change occurs in the scaffold and the quencher separates from the fluorophore. This conformation change removes the inhibitory effect of the quencher on the fluorophore, causing the generation of a detectable fluorescent signal that can be measured. In some embodiments, the fluorophore is any suitable fluorophore that produces a detectable signal, e.g., 6-carboxyfluorescein (6-FAM). In some embodiments, the quencher is any suitable quencher for use with the fluorophore, e.g., Black Hole Quencher 1® (BHQ-1) from Sigma Aldrich.

In some embodiments, oligonucleotide scaffold 102 lacks an explicit functional domain 112, i.e., is configured to merely bind to a target. However, as will be discussed is greater detail below, binding of oligonucleotide scaffold 102 can be sufficient to inhibit or deactivate a target by itself. Further, design of “inhibiting” scaffolds lacking the functional domains is achievable with minor changes to their “sensing” scaffold counterparts (with functional domains 112), e.g., by elongating elements of interior scaffold segments 104, exterior edge segments 106, or combinations thereof, to include features of the functional domain.

As discussed above, oligonucleotide scaffold 102 is designed to precisely arrange a plurality of binder insertion regions 108 in 1D, 2D, or 3D space. In some embodiments, oligonucleotide scaffold 102 can be of any suitable geometric shape, including, but not limited to, a binary conformation, a linear conformation, a branched conformation, a triangular conformation, a square conformation, a rectangular conformation, a pentagonal, hexagonal, heptagonal, octagonal, etc. conformation, a star conformation with a plurality of points, etc.

Referring again to the exemplary embodiment above, oligonucleotide scaffolds 102 according to the present disclosure can have a general configuration of a five-pointed star. In this embodiment, oligonucleotide scaffold 102 includes 25 oligonucleotides: five interior scaffold segments 104 and 20 exterior edge segments 106 (ten “edge” strands connected to interior scaffold segments 104, five “fix” strands stabilizing the scaffold, and five “close” strands capping the five tips of the “star.”) In this embodiment, oligonucleotide scaffold 102 includes SEQ. ID. NOs.: 1-5 as interior scaffold segments 104. In the absence of viral target, the interior scaffold segments 104 form stem loops. In some embodiments, oligonucleotide scaffold 102 includes SEQ. ID. NOs: 8-17 as “edge” exterior edge segments 106. In some embodiments, oligonucleotide scaffold 102 includes SEQ. ID. NOs.: 49-53 as “fix” exterior edge segments 106. In some embodiments, oligonucleotide scaffold 102 includes five SEQ. ID. NO.: 56 as “close” exterior edge segments. In some embodiments, the segment sequences are annealed to each other to form the generally shape of a five-pointed star.

In some embodiments, oligonucleotide scaffold 102 includes five functional domains 112. In some embodiments, the functional domains are composed of 5 fluorophore/quencher pairs. In some embodiments, fluorophore sequences include SEQ. ID. NOs:32, 34, 36, 38, 40. In some embodiments, a fluorophore, e.g., 6-FAM, is appended to the 5′ of the fluorophore sequences. In some embodiments, quencher sequences include SEQ. ID. NOs:33, 35, 37, 39, 41. In some embodiments, a quencher, e.g., BHQ-1, is appended to the 3′ of the quencher sequences. Oligonucleotide scaffold 102 can also be made without SEQ. ID. NOs.: 32-41 described above. In these, and other, embodiments, longer “edge” exterior edge segments 106, e.g., SEQ. ID. NOs.: 69-78 (8A-17A), can be substituted for SEQ. ID. NOs.: 8-17.

In some embodiments, oligonucleotide scaffold 102 includes a plurality of aptamers. In some embodiments, oligonucleotide scaffold 102 includes five aptamers attached to SEQ. ID. NOs.: 1-5 as interior scaffold segments 104. In some embodiments, the aptamers anneal to the interior scaffold segments at the 3′ ends of the interior scaffold segments. In some embodiments, oligonucleotide scaffold 102 includes five aptamers attached SEQ. ID. NOs: 8-17 as “edge” exterior edge segments 106. In some embodiments, the aptamers anneal to the interior scaffold segments at the 3′ ends of the interior scaffold segments. In some embodiments, the aptamers include SEQ. ID. NO.: 46. In some embodiments, the aptamers are attached to scaffold 102 via an overlap region, defined by SEQ. ID. NO.: 48 appended to the aptamer and SEQ. ID. NO.: 47 appended to the scaffold.

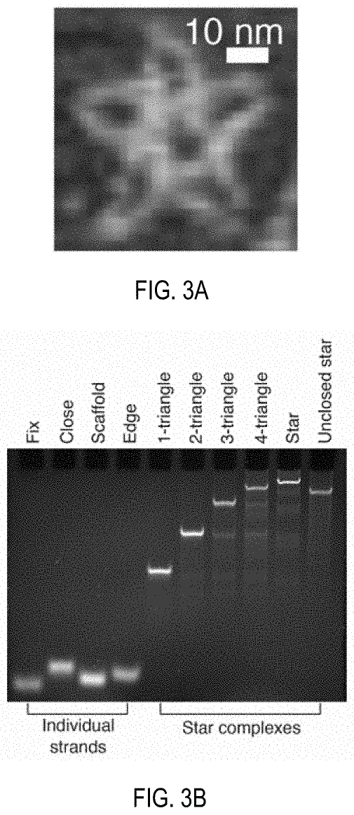

In this exemplary embodiment, each of the 10 external edges was 42-bp (base-pairs) long and connected to the internal edges through 4-arm junctions at the inner vertices. Each of the 5 internal edges carried a single-stranded DNA (ssDNA) region that can form a hairpin (stem-loop) structure with a 7-bp stem and 6-bp loop. When the hairpin unzips into ssDNA, it can stretch each internal edge to a distance that fits the distance between adjacent trivalent clusters. These hairpins provide molecular beacon-like motifs for viral detection and offer local structural flexibility to ensure binding of viral targets under various solution conditions and temperatures. Referring now to FIGS. 3A-3B, the formation of the complete DNA star was characterized by 4% non-denaturing polyacrylamide gel electrophoresis (PAGE) with one of each unique component strand and partial star complexes included for reference and Atomic force microscopy (AFM) imaging. AFM imaging confirmed formation of the star (FIG. 3A). For individual DNA nanostructure assemblies, non-denaturing gel electrophoresis is a valid and more reliable method for characterizing structural formation and yield. PAGE showed a distinct, major band corresponding to each of the 2D architectures, confirming that DNA star structures were formed with high efficiency (FIG. 3B).

The exemplary scaffold 102 was functionalized for DENV detection and inhibition by hybridizing a well-characterized, ED3-binding aptamer (SEQ. ID. NO.: 46) at each of the 10 vertices of the DNA star to form a star-aptamer complex that geometrically matched and targeted ED3 clusters. The aptamer exhibited weak binding to DENV as determined by surface plasmon resonance. However, binding strength increased as aptamers were placed onto scaffolds that increasingly matched the pattern of ED3 sites on DENV with the DNA star showing the greatest binding avidity. The fluorophore and quencher functional domain 112 hybridized to each inner edge strand flanking the hairpin made the scaffold a DENV sensor. Without wishing to be bound by theory, in the absence of DENV, FAM and BHQ-1 molecules were brought together by Watson-Crick base pairing in interior scaffold segments 104, much like a molecular beacon. Unlike a molecular beacon, however, which gives a fluorescent readout of target nucleic acid hybridization events, the interior scaffold segment hairpins are pulled apart and fully converted to single strands as a result of the binding of star aptamer complex to DENV surface ED3 clusters. This multivalent interaction, promoted by matched geometric aptamer-ED3 pattern, cause a separation of the FAM fluorophores from BHQ-1 quenchers to afford a fluorescent readout. The ssDNA region also offers local structural flexibility, allowing for retention of equivalent binding ability under perturbations by matching the scaffold aptamer pattern with slightly deformed arrangement of ED3 clusters due to temperature changes.

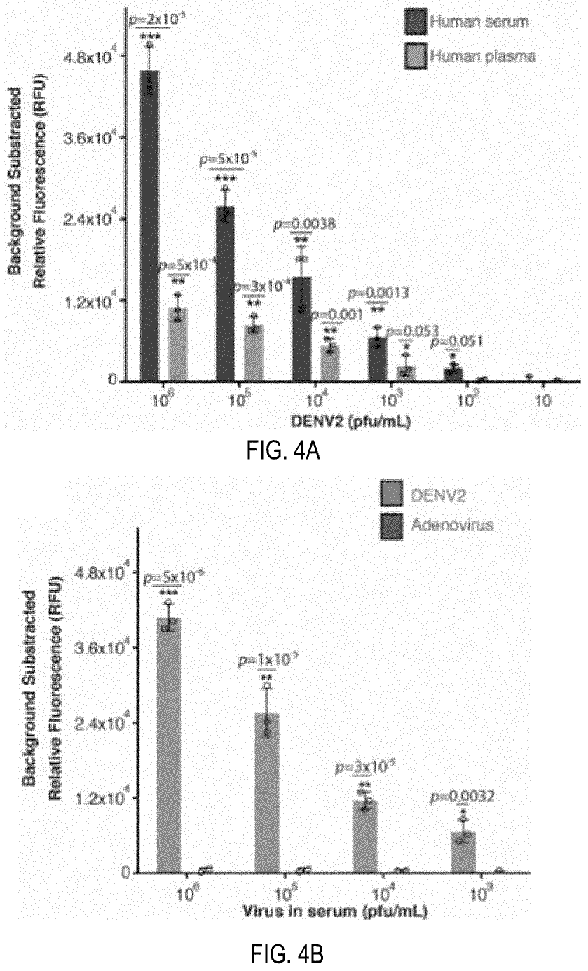

Referring to FIGS. 4A-4B, the DNA star-aptamer sensor was able to directly detect DENV virions with high sensitivity, affording a limit of detection (LoD) of 10² pfu/mL and 10³ pfu/mL, respectively in human serum and plasma (FIG. 4A). To exclude the possibility that the series of DENV-mixed scaffold sensing signals obtained within 1-2 minutes resulted from an automatic (non-target-triggered) separation of the fluorophores from quenchers, fluorescent readouts were measured over time at different temperatures. Fluorescent measurements over time showed that the FAM fluorophores remained quenched during the duration required for sensing and functioned at different temperatures. To examine the specificity of the scaffold, it was tested against adenovirus. Experimental results verify that the star-aptamer complex designed for DENV detection is not able to sense the adenovirus, even at high concentrations (FIG. 4B). To make a comparison between scaffold-based and gold standard methods under similar conditions, RT-qPCR and ELISA assays were carried out to respectively detect DENY RNA and NS1 antigen. The more sensitive method, RT-qPCR showed a LoD of 10³ pfu/mL after processing different concentrations of DENV-containing human serum, demonstrating the superior sensitivity of our new approach. The LoD of the scaffold is well below the viral concentration (>10⁵ pfu/mL) in patients on day [0], or the onset of illness, when fever and a variety of symptoms start to occur and the virus begins to become very pathogenic. Thus, detection before this date is highly beneficial to the patient and to the timely screening and control of pandemic outbreaks within surveillance and diagnostic networks.

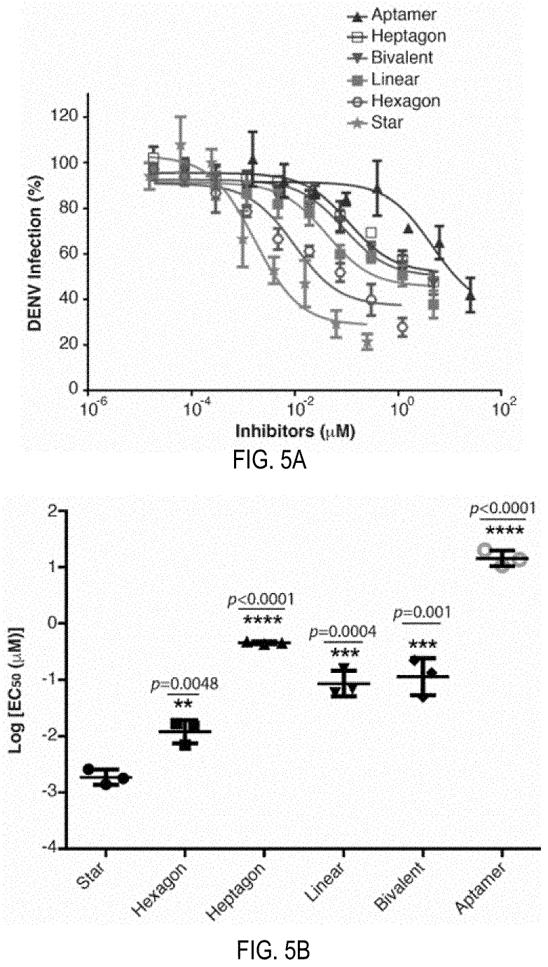

The in vitro inhibition of DENV through standard antiviral, plaque forming EC₅₀ assays was also examined. DENV viral particles were incubated with different concentrations of each inhibitor in human serum and the remaining infectivity was determined by a plaque reduction assay. The dose-dependent inhibition of DENV for the monovalent aptamer and each of the scaffold-aptamer complexes was examined (FIG. 5A). The EC₅₀ value (half maximal effective concentration) of the scaffold for DENV infection inhibition was 2 nM, whereas the EC₅₀ value of the monovalent aptamer was 15 μM (FIG. 5B). These results demonstrate that the scaffold multivalent inhibitor was 7.5×10³-fold more effective than the monovalent aptamer for the in vitro inhibition of DENV infection in human serum.

As discussed above, in some embodiments, oligonucleotide scaffolds 102 are multi-dimensional structures whose dimensions are determined by the combination of interior scaffold segments 104 and exterior edge segments 106. By way of further example, a hexagonal scaffold 102 for target sensing can include SEQ. ID. NOs.: 1-6 as interior scaffold segments 104, SEQ. ID. NOs. 18-29, 49-54, and 56-61 as exterior edge segments 106, and SEQ. ID. NOs.: 32-43 as functional domains 112. The hexagonal scaffold 102 can also be made without SEQ. ID. NOs.: 32-43. In these, and other, embodiments, longer “edge” exterior edge segments 106, e.g., SEQ. ID. NOs.: 79-90 (18A-29A), can be substituted for SEQ. ID. NOs.: 18-29. In some embodiments, a heptagonal scaffold 102 for target sensing can include SEQ. ID. NOs.: 1-7 as interior scaffold segments 104, SEQ. ID. NOs. 18-31, 49-55, and 56-62 as exterior edge segments 106, and SEQ. ID. NOs.: 32-45 as functional domains 112. The heptagonal scaffold 102 can also be made without SEQ. ID. NOs.: 32-45. In these, and other, embodiments, longer “edge” exterior edge segments 106, e.g., SEQ. ID. NOs.: 79-92 (18A-31A), can be substituted for SEQ. ID. NOs.: 18-31. In some embodiments, a bivalent scaffold 102 for target sensing can include SEQ. ID. NO.: 1 as interior scaffold segments 104, SEQ. ID. NOs. 63-64 as exterior edge segments 106, and SEQ. ID. NOs.: 32-33 as functional domains 112. The bivalent scaffold 102 can also be made without SEQ. ID. NOs.: 32-33. In these, and other, embodiments, longer “edge” exterior edge segments 106, e.g., SEQ. ID. NOs.: 93-94 (63A-64A), can be substituted for SEQ. ID. NOs.: 63-64. In some embodiments, a linear scaffold 102 for target sensing can include SEQ. ID. NOs.: 1-5 as interior scaffold segments 104, SEQ. ID. NOs. 63-68 as exterior edge segments 106, and SEQ. ID. NOs.: 32-41 as functional domains 112. The linear scaffold 102 can also be made without SEQ. ID. NOs.: 32-41. In these, and other, embodiments, longer “edge” exterior edge segments 106, e.g., SEQ. ID. NOs.: 93-98 (63A-68A), can be substituted for SEQ. ID. NOs.: 63-68.

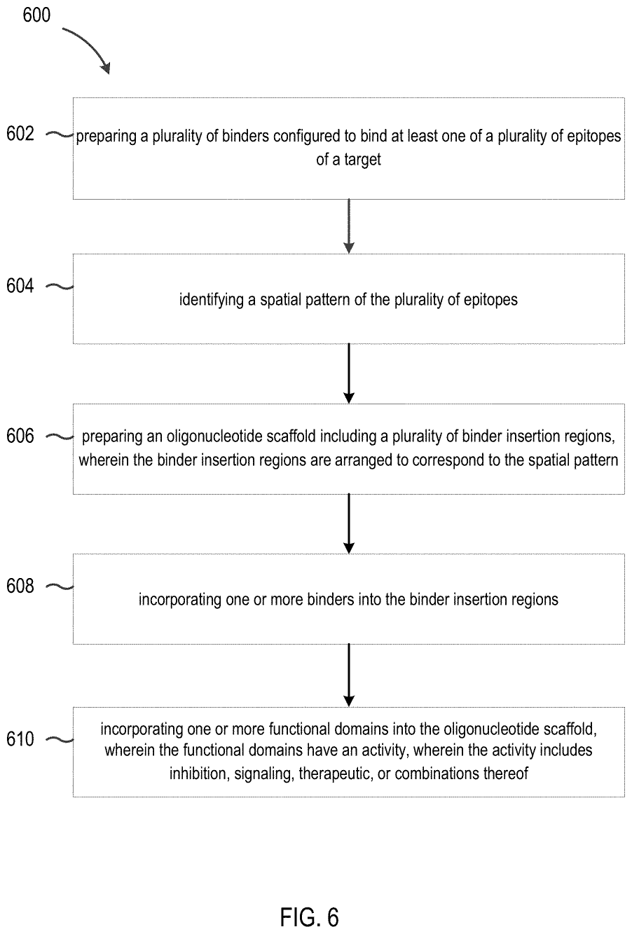

Referring now to FIG. 6, some embodiments of the present disclosure are directed to a method 600 of making a structure for pattern-recognized targeting of diseases. In some embodiments, at 602, a plurality of binders are prepared. As discussed above, the binders, e.g., peptides, aptamers, oligosaccharides, small molecules, or combinations thereof, are configured to bind at least one of a plurality of epitopes of a target. At 604, a spatial pattern of the plurality of epitopes is identified. At 606, an oligonucleotide scaffold is prepared. In some embodiments, the oligonucleotide scaffold includes a plurality of binder insertion regions. These binder insertion regions are arranged to correspond to the spatial pattern of epitopes identified on the target. At 608, one or more binders are incorporated into the binder insertion regions. In some embodiments, at 610, one or more functional domains are incorporated into the oligonucleotide scaffold. As discussed above, in some embodiments, the scaffold is prepared by annealing a plurality of oligonucleotide segments together to form a 1D, 2D, or 3D pattern. In some embodiments, the binders are incorporated into the scaffold by annealing with one or more of these oligonucleotide segments. In some embodiments, the functional domains are incorporated into the scaffold by annealing with one or more of these oligonucleotide segments.

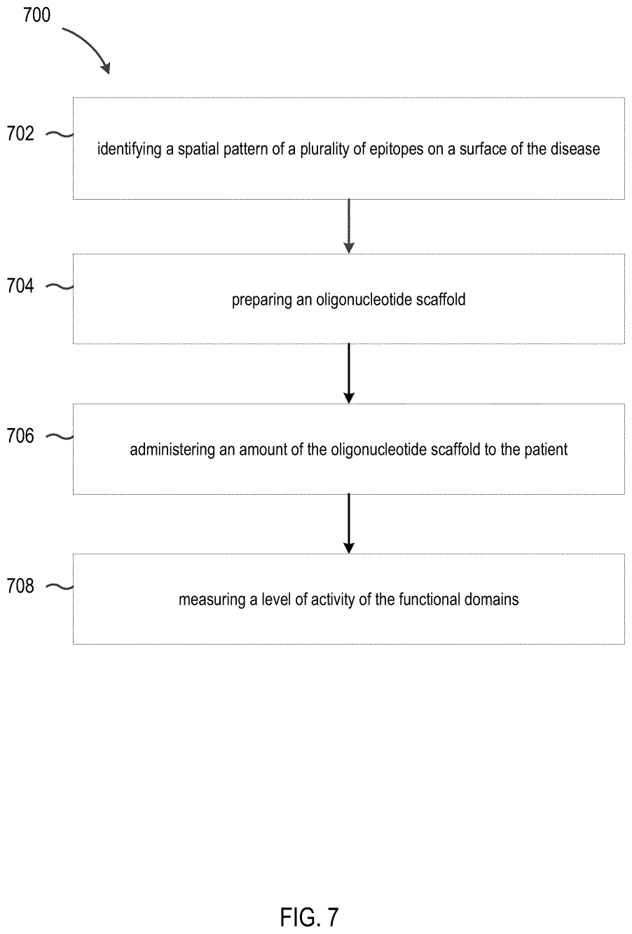

Referring now to FIG. 7, some embodiments of the present disclosure are directed to a method 700 of targeting a disease. In some embodiments, the disease is targeted in vivo, e.g., in a human patient. In some embodiments of method 700, at 702, a spatial pattern of a plurality of epitopes is identified on a surface of the disease. At 704, an oligonucleotide scaffold is prepared. As discussed above, in some embodiments, the scaffold is prepared by annealing oligonucleotide segments, e.g., one or more interior scaffold segments with one or more exterior edge segments, to define a structure. In some embodiments, the structure includes a plurality of binder insertion regions arranged to correspond to the spatial pattern of the disease epitopes. In some embodiments, a plurality of aptamers are annealed at the binder insertion regions, and one or more functional domains are annealed to the interior scaffold segments, the one or more exterior edge segments, or combinations thereof. As discussed above, the functional domains have a desired activity against the target disease, e.g., inhibition, signaling, therapeutic, or combinations thereof. In some embodiments, the oligonucleotide scaffold is included in a therapeutic or nutraceutic composition with one or more additives, e.g., pharmaceutically acceptable adjuvants, diluents, excipients, carriers, or combinations thereof. In some embodiments, the amount of the oligonucleotide scaffold is administered to a sample from the patient. In some embodiments, the therapeutic or nutraceutic composition includes one or more additional active ingredients.

In some embodiments, at 706, an amount of the oligonucleotide scaffold is administered to the patient. In some embodiments, at 708, a level of activity of the functional domains is measured.

### Methods

Sequences for all the scaffolds were designed using SEQUIN. All DNA oligonucleotides were purified by denaturing PAGE to remove partial products from synthesis. Human blood serum and plasma were obtained from Sigma-Aldrich. All chemicals for making necessary buffer solutions were obtained from Sigma-Aldrich and sterilized using a 0.22 μm filter from Millipore. The human dengue virus NS1 ELISA kit was obtained from Abbexa. Mica for AFM imaging was obtained from Ted Pella Inc. AFM probes were obtained from ScanAsyst-Air, Bruker Nano, Inc. SPR sensor CM5 chips were obtained from GE Life Sciences. Relevant stains, dyes, and reagents were obtained from Thermo-Fisher Scientific unless otherwise noted.

20% denaturing polyacrylamide gels were cast using 5 mL of 20% D-PAGE solution (20% acrylamide from 19 acrylamide to 1 bisacrylamide), 8.3 M urea, and 1×TBE buffer (89 mM Tris-HCl, 89 mM boric acid, 2 mM EDTA, pH 8.0), 7 μL TEMED, and 70 μL 10% APS per gel. Each gel can purify up to 4 ODs of DNA oligonucleotide mixed with 2× Denaturing Dye (8 M Urea, 10 mM NaOH, 1 mM EDTA, 0.25% (w/v) bromophenol blue, and 0.25% (w/v) xylene cyanol FF). DNA and Low Molecular Weight DNA Ladder (New England Biolabs) for size comparison was loaded onto the gels and run on a Mini-PROTEAN Tetra Vertical Electrophoresis Cell (Bio-Rad) at 450 V for 1-2 h. Each gel was then put into a stain box, filled with 50 mL of deionized water with 5 μL ethidium bromide and stained for 7 min with occasional shaking. Excess ethidium bromide was washed away with deionized water. Each gel was visualized under UV light to cut out bands of interest and eluted in 400 μL of elution buffer (0.5 M ammonium acetate, 1 mM EDTA, 10 mM Tris-HCl, pH=7.6) in a 1.5 mL Eppendorf tube at 4° C. After elution, 800 μL of butanol was added (creating an organic layer on top), vortexed, and removed three times. Sodium acetate (30 μL of 3 M solution) and 1 mL of 100% ethanol were added to remaining aqueous fraction. The solution was incubated on dry ice for 30 min and centrifuged for 30 min at 13,000 rpm at 4° C. to precipitate DNA. The supernatant was decanted and the remaining reconstituted in 50 μL of nuclease free water. The concentration of each DNA was measured using UV-Vis based on its absorbance at 260 nm.

A one-pot reaction was carried out to make the scaffolds by mixing all component strands in a final concentration 1×TAE-Mg²⁺—K⁺ buffer (pH=7.5), including 40 mM Tris-acetate, 2 mM EDTA, 12.5 mM Magnesium acetate, and 10 mM potassium chloride in Eppendorf 0.2 mL PCR Tube Strips (Fisher Scientific). Buffer were made from a 10× stock solution. All purified oligonucleotides were dissolved in nuclease free water. The final concentration of each DNA nanostructure was made at 0.4 μM. The mixture was annealed by incubating at 95° C. for 5 min followed by cooling from 95° C. to 25° C. at the rate of 0.15° C./min using a thermal cycler (TProfessional TRIO PCR Thermocycler). Scaffolds were made having equal ratios of interior scaffold segments and exterior edge segments, however, “close” segments were provided at 5:1 and aptamer segments at 10:1 the other segments. Partial scaffolds were made by following the same ratios listed for the full star but the species of each group were adjusted depending on number of triangles as elaborated below. After formation, all scaffolds were stored at 4° C. overnight for downstream use.

The following information includes the partial scaffolds used in the general format: “partial scaffold name” {“molar equivalence for each unique strand name-number” “SEQ. ID. NO.: XX)”}. 1-triangle {1 SEQ. ID. NO.: 1: 1 SEQ. ID. NOs.: 8-9: 1 SEQ. ID. NOs.: 49 and 53: 1 SEQ. ID. NO.: 56}; 2-triangle {1 SEQ. ID. NOs.: 1-2: 1 SEQ. ID. NOs.: 8-11: 1 SEQ. ID. NOs.: 49, 50, and 53: 2 SEQ. ID. NO.: 56}; 3-triangle {1 SEQ. ID. NOs.: 1-3: 1 SEQ. ID. NOs.: 8-13: 1 SEQ. ID. NOs.: 49-51 and 53: 3 SEQ. ID. NO.: 56}; 4-triangle {1 SEQ. ID. NOs.: 1-4: 1 SEQ. ID. NOs.: 8-15: 1 SEQ. ID. NOs.: 49-53: 4 SEQ. ID. NO.: 56}; and Unclosed star {1 SEQ. ID. NOs.: 1-5: 1 SEQ. ID. NOs.: 8-17: 1 SEQ. ID. NOs.: 49-53}. 1-triangle, 2-triangle, 3-triangle, 4-triangle, and the scaffolds were used for SPR experiments and functionalized with 3, 5, 7, 9, and 10 equivalents of ED3-binding aptamer, respectively. Each scaffold (with or without aptamer) was annealed by incubating at 95° C. for 5 min, and then cooling from 95° C. to 25° C. at the rate of 0.15° C./min using a thermal cycler (TProfessional TRIO PCR Thermocycler). After formation, each scaffold was stored at 4° C. overnight for downstream use. For SPR, scaffolds were diluted to the normalized aptamer concentration as needed.

The scaffolds (without aptamer) were characterized by 4% non-denaturing PAGE in 1×TAE-Mg²⁺ buffer (40 mM Tris-acetate, 2 mM EDTA, and 12.5 mM Magnesium acetate, pH=7.5). The DNA bivalent or flexible linear (linear) scaffold was characterized by 10% non-denaturing PAGE in 1×TAE-Mg²⁺ buffer. Each gel was run at a constant voltage of 10 V/cm for 2 h, stained by SYBR Green (Thermo Fisher), and scanned by GelDoc (Bio-Rad). Intensity of each DNA species on the gel was quantified by Image Lab (Bio Rad) or ImageJ.

For AFM imaging, APTES ((3-Aminopropyl) triethoxysilane, 20 μL 0.5%) was added onto freshly cleaved mica (Ted Pella Inc.) surface and was incubated for 2 min to increase the adsorption of the folded DNA scaffolds (without aptamer). The mica surface was then washed by ddH₂O and dried by compressed air. A drop of 5 μL of the 5 nM DNA scaffold-including solution was deposited onto pre-treated mica surface and incubated for 5 min. 40 μL of 1×TAE-Mg²⁺ buffer was further added. Imaging was performed under tapping mode in the fluid cell on Multimode AFM with SNL-10 probe (Bruker Nano, Inc.).

For SPR analysis, purified DENV serotype 2 viral particles were immobilized onto a research grade CM5 SPR chip (GE healthcare, Uppsala, Sweden) according to a standard amine coupling protocol. Briefly, carboxymethyl groups on CM5 chip surface were first activated using an injection pulse of 35 mL (flow rate 5 μL/min) of an equimolar mix of N-ethyl-N-(dimethyaminopropyl) carbodiimide (EDC) and N-hydroxysuccinimide (NHS) (final concentration of 50 μM, mixed immediately before injection). Following the activation, each virus was prepared at 10¹⁰ plaque forming units (pfu) per mL in a 100 mM sodium acetate (pH 5.0) buffer and injected over the activated biosensor surface. The successful immobilization of the virus particles was confirmed by the observation of a 2000 resonance unit (RU) increased baseline signal. Excess unreacted carboxymethyl groups on the sensor surface were deactivated with a 35 μL injection of 1 M ethanolamine. A reference flow cell was prepared using the same procedures of amine coupling (activation/deactivation) except no viral particles were immobilized. Different dilutions of each DNA-aptamer complex were injected over the virus chip at a flow rate of 50 μL/min with 1×TAE-Mg⁺²—K⁺ as running buffer. At the end of the sample injection, the 1×TAE-Mg⁺²—K⁺ was flowed over the sensor surface to facilitate dissociation. After a 3 min dissociation, the sensor surface was fully regenerated by injecting 50 of 2 M NaCl. The SPR response (sensorgram) was monitored as a function of time at 25° C. SPR measurements were performed on a BIAcore 3000 (GE healthcare, Uppsala, Sweden) operated using the BIAcore 3000 control software. Each sample was measured by three independent experiments. The resulting sensorgrams were used for binding kinetics parameter determination (i.e., association rate constant: kₐ; dissociation rate constant: k_{d}; and binding equilibrium dissociation constant: K_{D}, K_{D}=k_{d}/kₐ) by globally fitting the entire association and dissociation phases using 1:1 Langmuir binding model from BiaEvaluation software 4.0.1 (GE healthcare, Uppsala, Sweden).

Scaffolds or control sensors in solution (10 μL at 0.4 μM) were added per well in PurePlus PCR Tube Strips (Thomas Scientific) closed with MicroAmp Optical 8-Cap Strips (Applied Biosystems) before 10 μL human serum or plasma solution was added to each tube for sensing; DENV2 (New Guinea-C, NGC) or RFP Adenovirus (VECTOR BIOLABS) were spiked at different concentrations (pfu/mL) in human serum or plasma beforehand. A fluorometer was used to quantify the fluorophore intensity of DNA star and differently shaped control sensors. Fluorophore (6-FAM) intensities were observed at different temperatures (4-23° C.) on the StepOnePlus Real-Time PCR System (ThermoFisher Scientific) to show that sensor function and aptamer binding was robust at low temperatures. Similar values were observed at room temperature and can also be recorded using a portable fluorometer. The program was set to read the sample first at 30 s, again at 45 s, and finally at 60 s. It took less than 5 min to mix and read the sample. Background fluorescence was determined by mixing 10 μL of sensor with 10 μL of human serum that included no virus. Background fluorescence was subtracted from the fluorescence observed in the sample including 10 μL of sensor mixed with 10 μL for each concentration of virus-spiked human serum.

For RT-qPCR based sensing, the time-to-results was about 4 h. The MagMAX-96 Viral RNA Isolation Kit (ThermoFisher Scientific) was used to extract the RNA sample and was further processed by a MagMAX Express-96 Deep Well Magnetic Particle Processor (ThermoFisher Scientific). This RNA extraction step took at least 30-45 min. A 50 μL aliquot of each viral concentration was then used for extraction and eluted into 50 μL. Eluted RNA (5 μL) was used for the following steps. The 5 μL volume was used since the original sample used for this method had a concentration (pfu/mL) double that of the sample used for the DNA nanostructure-based sensing. The qScript XLT 1-step RT-qPCR ToughMix (QuantaBio) on the Applied Biosystem 7500 real-time PCR system was employed for RT-qPCR assay, which takes about 45 min to 1 h. The program used was 5 min at 50° C. (for the reverse transcriptase reaction), 10 min at 95° C. (to inactivate reverse transcriptase and activate the hot-start PCR), then 40 cycles consisting of 3 s at 95° C. and 30 s at 60° C. was performed. Amplification plots for each RNA sample were generated to determine the LoD of RT-qPCR-based method. The probe and primers used for DENV 2 (BALI-TRV-005 isolate) are targeting the envelope protein gene. The forward primer: SEQ. ID. NO.: 99, the reverse primer: SEQ. ID. NO.: 100, and the qPCR probe: [[5′-6-]]FAM-SEQ. ID. NO.: 101-BHQ-1.

The time-to-results for ELISA-based sensing was about 3-4 h. Cell culture supernatants samples including certain concentrations (pfu/mL) of DENV virions were centrifuged at 2,500 rcf for 20 min to remove precipitant. A Human Dengue Virus NS1 (DV NS1) ELISA Kit (Abbexa) was then used to quantify NS1 level in each of the samples. More specifically, each sample was diluted 1:5 (10 μL of each sample with 40 μL of dilution buffer). Concentrated wash buffer was diluted with distilled water (20 mL of concentrate was diluted into 580 mL of distilled water). A 50 μL aliquot of the negative and positive controls and the diluted samples were transferred onto test sample wells and mildly mixed. After adding the samples, the plate was covered and incubated at 37° C. for 30 min before the solution was discarded. The plate was washed 5-times with diluted wash buffer (300 μL per well) using a multi-channel pipette. The plate was then inverted and blotted against clean paper towels after the final wash. HRP conjugate reagent (50 μL) was added to each well. The plate was covered and incubated at 37° C. for another 30 min before the solution was discarded. The plate was then washed 5 times with diluted wash buffer (300 μL per well) using a multi-channel pipette. For the next step of the assay, 50 μL of TMB Substrate A and 50 μL of TMB Substrate B were aliquoted into each well. The plate was gently shaken by hand for 30 s before incubation at 37° C. for 15 min and covered from light. Finally, 50 μL of stop solution was added to each well and the absorbance of each well was measured at 450 nm. The cut-off value was 0.15 absorbance units greater than the negative control.

For the in vitro inhibition of DENV (EC₅₀ and plaque assays), vero cells were seeded at a density of 3×10⁵ cells/3 mL MEM culture medium (with 10% FBS) in each well of a 6-well plate 72 h prior to infection. Approximately 200 plaque forming units of DENV serotype 2 (DENV2 NGC), after dilution in Hank's Balanced Salt Solution (HBSS) (GIBCO, Grand Island, N.Y.) including 0.4% (w/v) bovine albumin fraction V (BSA fraction V, GIBCO), were incubated at a 1:1 ratio in human serum (with or without an inhibitor). The mixtures were incubated in a 5% CO₂ incubator at 37° C. for 1 h. Then virus-inhibitor or virus-control mixtures were used directly to infect the confluent monolayer of Vero cells or diluted in MEM+2% FBS before being added onto the Vero cell monolayers. The inoculum was 100 μl/well. The virus absorption was done in a 5% CO₂ incubator at 37° C. for an additional hour. Then, the inoculated mixtures were removed and the cells were washed once with 5 mL of phosphate buffered saline (PBS) before adding 3 mL of agarose overlay medium (lx MEM, 5% FBS, 0.6% Oxoid purified agar) as a standard condition for the in vitro anti-viral assay. After 4-5 days of incubation, the plaques were stained with a 2^{nd} overlay including 1×MEM, 1% FBS, 0.6% Oxoid purified agar and 0.0067% (w/v) Neutral Red. After continuous incubation at 37° C. for 24-48 h, plaques were examined and counted. Nonlinear regression (the Hill equation to dose-response curves) was used to calculate EC₅₀ values. Each test sample was measured by three independent experiments. The statistical interpretation of the data and EC₅₀ values were expressed as 95% confidence intervals.

Time-lapsed imaging assay of viral cell entry or inhibition. Alexa Fluor 594 NHS Ester (100 μM) was dissolved in 0.2 M sodium bicarbonate buffer (pH 8.3) immediately before labeling and addition of DENV (DENV2 NGC) in sodium bicarbonate buffer at final concentrations of 50 μM dye. The mixture was incubated at room temperature for 1 h with constant gentle inversions. The labeling reaction was quenched by adding freshly prepared 1.5 M hydroxylamine (pH 8.5) and incubated at room temperature for 1 h. Alexa 594-labeled DENV was purified by sequentially using Microcon DNA Fast Flow Centrifugal Filter (Millipore-Sigma) and Sephadex G-25 columns (Amersham, GE Healthcare) to remove the excess dye. HepG2 (ATCC HB-8065) cells were cultured in a glass bottom cell culture dish (diameter (1) 20 mm, MatTek) for 24 h at 37° C. in 5% CO2. The cells (1 mL in DMEM/FBS medium) were stained with Hoechst 33342 solution (10 μM, Thermofisher). Alexa 594-labeled DENV (5×10⁴ pfu) was added followed by time-lapsed imaging assay with a confocal microscopy (Leica TCS SP8 STED Microscope). The scaffold-bound DENV (5×10⁴ pfu) was also subjected to the time-lapsed imaging assay with the same confocal microscopy. For the multicolor experiments, HepG2 cells were labeled with both Vybrant™ Dil (5 μM, Thermofisher) and Hoechst 33342 solution (2 μM) for membrane and nucleus staining, respectively, and cells were washed three times with DMEM. DENV was stained with Vybrant™ DiD followed by previous protocol. After adding DiD-labeled DENV (or scaffold-bound DENV stained with DiD) into the HepG2 cells stained with both Dil and Hoechst 33342, imaging was performed using confocal microscopy and four fluorescence wavelengths, 461 (nucleus, blue), 488 (DNA star, green), 565 (cell membrane, yellow), and 665 nm (DENV, red), were detected simultaneously. Image analyses were performed using Imaris software (Bitplane).

Methods and system of the present disclosure are advantageous to detect and inhibit viruses and toxins with high specificity. The multivalent interaction and spatial pattern recognition of the DNA oligonucleotides confers selectivity by achieving high binding avidity, leading to significantly potent viral sensing and inhibition. These strategies can be adapted to combat other disease-causing entities by generating the requisite ligand patterns. The designer DNA scaffold may contribute to the basic knowledge of virus and bacteria structure and biology, particularly as a tool to investigate the molecular targeting of disease-host cell interactions and placental crossing by providing a better understanding of ligand spacing in a more complex space.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A structure for pattern-recognized targeting of diseases, comprising:
an oligonucleotide scaffold including a plurality of binder insertion regions, wherein the binder insertion regions are arranged in a pattern conforming to a plurality of epitopes of a target; and
a plurality of binders incorporated into the binder insertion regions, wherein the binders are configured to bind at least one of the plurality of epitopes.

2. The structure according to claim 1, further comprising one or more functional domains having an activity, wherein the activity includes inhibition, signaling, therapeutic, or combinations thereof.

3. The structure according to claim 2, wherein the functional domains exhibit a first activity when unbound and a second activity when bound to the plurality of epitopes.

4. The structure according to claim 3, wherein the functional domains include a hybridized fluorophore and a quencher, wherein the quencher inactivates the fluorophore as the first activity, and wherein the quencher separates from the fluorophore upon binding of the plurality of binders to generate a detectable fluorescent signal as the second activity.

5. The structure according to claim 1, wherein the binders include peptides, aptamers, oligosaccharides, small molecules, or combinations thereof.

6. The structure according to claim 1, wherein the target is Dengue virus, Zika virus, influenza virus, adenovirus, bacterial toxin, or combinations thereof.

7. The structure according to claim 1, wherein the scaffold includes one or more interior scaffold segments and one or more exterior edge segments.

8. The structure according to claim 7, wherein the one or more exterior edge segments include SEQ. ID. NO.: 8-45S, 49-68A, or combinations thereof.

9. The structure according to claim 7, wherein the interior scaffold segments include a stem-loop structure.

10. The structure according to claim 9, wherein the interior scaffold segments include SEQ. ID. NO.: 1-7, or combinations thereof.

11. The structure according to claim 7, comprising:
five aptamers attached to interior scaffold segments, wherein the aptamers attach to the interior scaffold segments at the 3′ ends of the interior scaffold segments; and
five aptamers attached to exterior edge segments, wherein the aptamers attach to the exterior edge segments at the 3′ ends of the exterior edge segments.

12. The structure according to claim 7, wherein the interior scaffold segments and exterior edge segments are arranged in a 3D pattern.

13. A method of making a structure for pattern-recognized targeting of diseases, comprising:
preparing a plurality of binders configured to bind at least one of a plurality of epitopes of a target;
identifying a spatial pattern of the plurality of epitopes;
preparing an oligonucleotide scaffold including a plurality of binder insertion regions, wherein the binder insertion regions are arranged to correspond to the spatial pattern; and
incorporating one or more binders into the binder insertion regions.

14. The method according to claim 13, further comprising incorporating one or more functional domains into the oligonucleotide scaffold, wherein the functional domains have an activity, wherein the activity includes inhibition, signaling, therapeutic, or combinations thereof.

15. The method according to claim 13, wherein the binders include peptides, aptamers, oligosaccharides, small molecules, or combinations thereof.

16. The method according to claim 13, wherein the target is Dengue virus, Zika virus, influenza virus, adenovirus, bacterial toxin, or combinations thereof.

17. The method according to claim 13, wherein preparing an oligonucleotide scaffold including a plurality of binder insertion regions includes:
annealing one or more interior scaffold segments with one or more exterior edge segments, wherein the interior scaffold segments include a stem-loop structure.

18. The method according to claim 17, further comprising:
annealing five aptamers to the interior scaffold segments, wherein the aptamers attach to the interior scaffold segments at the 3′ ends of the interior scaffold segments; and
annealing five aptamers to the exterior edge segments, wherein the aptamers attach to the exterior edge segments at the 3′ ends of the exterior edge segments.

19. A method of targeting a disease in a patient, comprising:
identifying a spatial pattern of a plurality of epitopes on a surface of the disease;
preparing an oligonucleotide scaffold including:
annealing one or more interior scaffold segments with one or more exterior edge segments to define a structure including a plurality of binder insertion regions, wherein the binder insertion regions are arranged to correspond to the spatial pattern;
annealing a plurality of aptamers at the binder insertion regions; and
annealing one or more functional domains to the interior scaffold segments, the one or more exterior edge segments, or combinations thereof, wherein the functional domains have an activity, wherein the activity includes inhibition, signaling, therapeutic, or combinations thereof;
administering an amount of the oligonucleotide scaffold to the patient; and
measuring a level of activity of the functional domains.

20. The method according to claim 19, wherein the target is configured to bind to SEQ. ID. NO.: 46.
